(19) United States
(12) Patent Application Publication
(10) Pub. No.: US 2009/0099133 A1
(43) Pub. Date: Apr. 16, 2009

(54) Isoquinolines Derivatives as Igf-1R Inhibitors

(76) Inventors: Jan Gunzinger, Couvet (CH); Kurt Leander, Peseux (CH)

Correspondence Address: HARNESS, DICKEY & PIERCE, P.L.C., P.O. BOX 8910, RESTON, VA, 20195, US

(21) Appl. No.: 11/991,531

(22) PCT Filed: Sep. 8, 2006
(86) PCT No.: PCT/IB2006/002474

§ 371 Date: Apr. 15, 2008

Related U.S. Application Data
(60) Provisional application No. 60/715,170, filed on Sep. 9, 2005.

(30) Foreign Application Priority Data
Sep. 9, 2005 (IB) PCT/IB2005/002667

(51) Int. Cl. A61K 31/675; C07D 217/02; A61P 29/00

(52) U.S. Cl. 514/82; 546/144

(57) ABSTRACT

Compounds of the formula (I) were synthesized. They were found to down-regulate or inhibit the expression or function of the IGF-1 receptor.

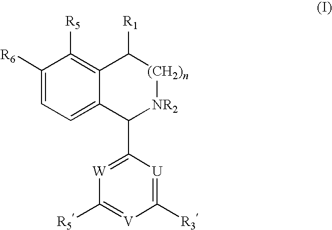

### FIELD OF THE INVENTION

The present invention relates to novel compounds and prodrug compounds capable of down-regulating or inhibiting the expression or function of the insulin-like growth factor-1 receptor (IGF-1R). The invention is also directed to pharmaceutical compositions and methods of down-regulating or inhibiting IGF-1R expression or function in order to prevent and/or treat cancer and other abnormal cell growth, and metabolic as well as blood vessel proliferate disorders, in which uncontrolled expression of this receptor is observed.

### BACKGROUND ART

The present invention is an improvement of some aspects over PCT/CH2004/000147 from the same applicant, the content of which is incorporated herein by reference in its entirety.

The insulin-like growth factor receptor (IGF-1R) is one of 58 trans-membrane tyrosine kinase receptors present in humans [Review: Structure and function of the Type 1 insulin-like growth factor receptor. T. E. Adams et al. Cell. Mol. Life. Sci. 57 (2000) 1050-1093; Insulin-Like Growth Factors. Kluwer Academic/Plenum Publishers (2003). Editors: LeRoith, D., Zumkeller, W. and Baxter, R. C.]. Genetic evidence and studies on cells lacking the IGF-1 receptor have demonstrated that it is required for optimal growth, but not an absolute condition for growth [Baserga et al. Biochim. Biophys. Acta 1332 (1997) 105-126]. An expression of the IGF-1 receptor protects cells from apoptosis and seems to be a requirement for the establishment and maintenance of the transformed phenotype both in vitro and in vivo [R. Baserga et al. Biochim. Biophys. Acta 1332 (1997) 105-126]. Several in vitro and in vivo studies have demonstrated that inhibition of the expression or function of the IGF-1 receptor reverses the transformed phenotype and inhibits tumour cell growth. The techniques used in these studies include neutralizing antibodies [Kalebic et al. Cancer Res. 54 (1994) 5531-5534; Arteaga, C. L. et al. Cancer Res. 49 (1989) 6237-6241; De Leon, D. D. et al. Growth Factors 6 (1992) 327-336], antisense oligonucleotides [Resnicoff et al. Cancer Res. 54 (1994) 2218-2222; Andrews, D. W. et al. J. Clin. Oncol. 19 (2001) 2189-2200; White, P. J. et al. Antisense Nucleic Acid Drug Dev. 10 (2000) 195-203], dominant negative mutants [D'Ambrosio et al. Cancer Res. 56 (1996) 4013-4020; Prager, D. et al. Proc. Natl. Acad. Sci. USA 91 (1994) 2181-2185; Reiss, K. et al. Clin. Cancer Res. 4 (1998) 2647-2655], triple-helix forming oligonucleotides [Rinninsland et al. Proc. Natl. Acad. Sci. USA 94 (1997) 5854-5859], antisense mRNA [Nakamura et al. Cancer Res. 60 (2000) 760-765] and RNA interference using a double stranded RNA [V. M. Macaulay et al. WO-A-03/100059].

The use of antisense oligonucleotides to inhibit the IGF-1 receptor expression in keratinocytes has been shown to reverse the epidermal hyper proliferation in psoriasis lesions [C. J. Wraight et al. Nat. Biotechnol. 18 (2000) 521-526].

Down-regulation of the IGF-1 receptor would possibly also have beneficial effect with respect to diseases such as diabetic retinopathy [L. K. Shawver et al. DDT 2 (1997) 50-63] as well as atherosclerosis, restinosis [A. Bayes-Genis et al. Circ. Res. 86 (2000) 125-130] and rheumatoid arthritis [J. Pritchard et al. J. Immunol. 173 (2004) 3564-3569].

The IGF-1 receptor system is regarded as an attractive target in the prevention and/or treatment of diseases that are dependant on an expression or over-expression of the IGF-1 receptor for their proliferation [L. Long et al. Cancer Research 55 (1995) 1006-1009, R. Baserga TIBTECH 14 (1996) 150-152; R. Baserga et al. Endocrine 7 (August 1997) 99-102; V. M. Macaulay et al. Annals of Oncogene 20 (2001) 4029-4040; A. J. Salisbury et al. Horm. Metab. Res. 35 (2003) 843-849; Mitsiades, C. S. et al. Cancer Cell 5 (2004) 221-230].

A series of substances, named tyrphostins, have been claimed to down-regulate or inhibit the expression of the IGF-1 receptor [M. Parrizas et al. Endocrinology 138 (1997) 1427-1433; G. Blum et al. Biochemistry 39 (2000) 15705-15712; G. Blum et al. J. Biol. Chem. 278 (2003) 40442-40454]. The drawback with the tyrphostins are their low activity in cell systems and that they cross-react with the insulin receptor.

It has been demonstrated [L. Kanter-Lewensohn et al. Mol. Cell. Endocrinology 165 (2000) 131-137] that tamoxifen, at high concentration, has the ability to down-regulate or inhibit the tyrosine phosphorylation of the IGF-1R β-subunit, thereby blocking downstream signalling.

In U.S. Pat. No. 6,337,338 B1; a number of heteroaryl-aryl urea substances are described as antagonists of the IGF-1 receptor. In cell growth inhibition studies on MCF-7 and MCF-10 cell lines the substances showed low activities.

In the patent application WO 02/102804 A1 it is demonstrated that podophyllotoxin, deoxypodophyllotoxin, picropodophyllin and deoxypicropodophyllin are selective and efficient inhibitors of the IGF-1 receptor. Deoxypicropodophyllin has previously [A. Akahori et al. Chem. Pharm. Bull. 20 (1972) 1150-1155] been shown to be superior to deoxypodophyllotoxin in retarding the death of mice inoculated with lymphatic leukemia L1210. No mechanism of action, however, was proposed.

In the patent application WO 02/102805 A1 it is shown that also acetylpodophyllotoxin, epipodophyllotoxin, podophyllotoxone and 4′-demethylpodophyllotoxin are potent inhibitors of the IGF-1R phosphorylation.

In the patent application WO 03/048133 A1 a number of pyrimidine derivatives are described as modulators of the IGF-1 receptor. However, these pyrimidine derivatives have shown a poor IGF-1R down-regulating activity.

PCT/CH2004/000147 (Analytecon S. A.) provides new heterocyclic compounds with surprisingly improved IGF-1R down-regulating activity.

There is, however, still a need for IGF-1R down-regulating compounds as alternatives to those described in PCT/CH2004/000147 and elsewhere, with e.g. improved aqueous solubility as well as different physical and metabolic properties. In addition, there is also a need for prodrugs which provide anti-cancer agents, such as the compounds described in PCT/CH2004/000147 and herein, with improved water solubility, a high systemic uptake of the prodrug or the active parent compound after oral administration and in some particular cases a sufficiently extended plasma half-life time to maintain in vivo concentration in a therapeutic range for a prolonged period of time.

The present invention aims at providing new compounds with high IGF-1R down-regulating activity, wherein the above-identified problems are successfully solved. In particular, the invention also aims at providing a pharmaceutical prodrug composition, wherein water solubility, stability and the like of the active parent compounds described herein and in PCT/CH2004/000147 are improved.

### SUMMARY OF THE INVENTION

The object set is achieved by the compounds of the following formula (I):

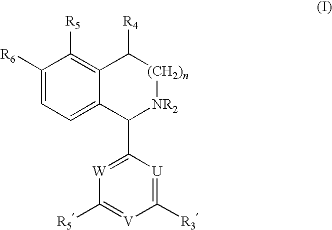

wherein

R₄ designates H; OH; CN; trifluoromethyl; NH₂; NHCN; NHCOCH₃; NHCOCH₂CH₃; NHCHO; NHCOOCH₃; amino(C₁-C₆) alkyl; amino(C₁-C₃)dialkyl; (C₁-C₆)alkoxy; (C₁-C₆)alkyl; carbonyl-R₉ wherein R₉ designates hydrogen, (C₇-C₆)alkyl, (C₁-C₆)alkoxy; (C₁-C₆)alkyl-R₁₀; (C₁-C₆)alkoxy-R₁₀; amino(C₁-C₆)alkylR₁₀ and amino(C₃-C₃)dialkyl-R₁₀ whereby R₁₀ designates at least one OMe, OEt, OPr, OIsopropyl, OH, CN, NH₂, ester groups with (C₁-C₃)alkyl, carbonate groups with (C₁-C₃)alkyl;

R₂ designates hydrogen, Me, Et, CHO, CN, OH, OMe, COR₉, COOR₉, CONHR₉ or CSNHR₉, whereby R₉ denotes (C₁-C₄)alkyl;

R₅ designates hydrogen, (C₁-C₄)alkyl, OH, (C₁-C₄)alkoxy, (C₁-C₂)alkoxy partly or fully fluorinated, trifluoromethyl, halogen or OX;

R₆ designates Me, halogen, hydrogen, (C₁-C₄)alkoxy, (C₁-C₂)alkoxy partly or fully fluorinated, SMe or SEt; if R₅ is OH or OX, R₆ may be hydrogen;

n is 1 or 2;

R₃′ and R₅′ each independently designate OH, Me, Et, OMe, OMe partly or fully fluorinated, trifluoromethyl or halogen;

U designates N or CR₂′, whereby R₂′ denotes hydrogen, (C₁-C₄)alkyl, (C₁-C₄)alkoxy, trifluoromethyl or halogen;

V designates N or CR₄′, whereby R₄′ denotes hydrogen, (C₁-C₆)alkoxy, (C₁-C₄)alkoxy partly or fully fluorinated, (C₁-C₆)alkyl, OH, trifluoromethyl, halogen or OX;

W designates N or CR₆′, whereby R₆′ denotes hydrogen, (C₁-C₄)alkyl, (C₁-C₄)alkoxy, trifluoromethyl or halogen;

wherein OX designates a group capable of conferring a prodrug property; and pharmaceutically acceptable salts thereof, where applicable (see below).

Also provided are the prodrug compounds having the following general formula (II):

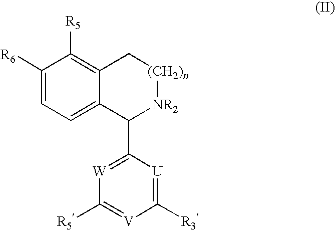

wherein at least one OX group is present in R₅ and/or R₄′ thereby conferring a prodrug property to the compound of formula (II); and pharmaceutically acceptable salts thereof.

Alternatively, OX groups may be present in both R₅ and R₄′ (when V designates CR₄′).

Preferred embodiments of the prodrug compounds (II) are derivable from the following description.

Further objects of the invention are the use of the compounds (I) and/or prodrug compounds (I) in the manufacture of a medicament, particularly for the prevention or treatment of diseases in which the down-regulation or inhibition of the expression or function of the IGF-1 receptor is considered beneficial and pharmaceutical compositions containing the same.

Other objects and advantages will become apparent to those skilled in the art from a review of the ensuing detailed description, which proceeds with reference to the following drawings, and the attendant claims.

### BRIEF DESCRIPTION OF THE FIGURE

FIG. 1 shows the rate of dephosphorylation of (1R)-1-(3,4,5-trimethoxyphenyl)-2-formyl-5-(dihydrogen phosphate)-6-methoxy-1,2,3,4-tetrahydroisoquinoline by alkaline phosphatase.

### DETAILED DESCRIPTION OF THE INVENTION

For the purposes of the present invention, a “prodrug” is an entity which either comprises an inactive form of an active drug (parent compound) or includes a chemical group which confers preferred characteristics on the drug. In other words, the invention concerns a composition which has the potential of producing a desired physiological effect on cells, but is initially inert (i.e. does not produce said effect), and only after undergoing some modifications becomes physiologically active and produces said physiological effect on cells. In particular, the derivative of a parent compound of the present invention has a chemically or metabolically degradable group, and becomes pharmaceutically active after biotransformation.

Biotransformation of the prodrug or a salt thereof, according to the invention is carried out under physiological conditions (in vivo) and is a result of a reaction with an enzyme, or a body fluid such as gastric acid, blood etc., thus undergoing an enzymatic oxidation, reduction, hydrolysis etc. or a chemical hydrolysis to convert into the active parent compound.

As used herein, the terms “parent compounds” or “active parent compounds” or “active drugs” are used interchangeably herein to designate the heterocyclic compounds described herein and in PCT/CH2004/000147 (Analytecon S. A.) and lacking moiety OX.

The term “physiological effect” concerns any effect a drug may have on cells, in order to improve the health of the subject administered with the drug. The effect is produced in order to treat, prevent a disease, a defect or pathological condition or to alleviate some of the manifestations of a disease, defect or pathological condition.

The term “comprise” is generally used in the sense of include, that is to say permitting the presence of one or more features or components.

The compounds (I) and the prodrug compounds of formula (II) contain a tetrahydroisoquinoline moiety (n=1) or a tetrahydrobenzazepine moiety (n=2).

In the above formula (I) preferably R₄ is H, OH, NH₂, amino(C₁-C₃), amino(C₁-C₃)dialkyl, CH₂OH, COOCH₃, OCOOCH₃, methyl, Et and the like.

Preferably R₂ is Me, OH, CN, CHO, COR₉ or COOR₉; particularly preferred examples of R₂ are Me (methyl), CHO (formyl), COMe (acetyl) and CN (cyano).

Preferably R₅ is hydrogen, OH, Me, OMe, halogen or OX; and preferably R₆ is OCHF₂, OMe, OCH₂CF₃ or OEt. Particularly preferably R₅ is hydrogen, OX, OH or OMe and R₆ is OCHF₂, OMe OCH₂CF₃ or OEt. The most preferred substituent pattern for R₅ and R₆ is R₅=hydrogen, OH or OX and R₆═OCHF₂, OMe, OCH₂CF₃ or OEt.

In formula (I) the substituent on the 1-position of the 1,2,3,4-tetrahydroisoquinoline or 2,3,4,5-tetrahydro-1H-2-benzazepine moieties may be a phenyl substituent (U=CR₂′; V═CR₄′; W═CR₆′), a 4-pyridyl substituent (U═CR₂′; V═N; W═CR₆′), a 2-pyridyl substituent (V═CR₄′; U═N, W═CR₆′, or U═CR₂′, W═N), a 2-pyrimidyl substituent (U, W═N; V═CR₄′), a 4-pyrimidyl substituent (V═N; U═CR₂′, W═N, or U═N, W═CR₆′), or a triazinyl substituent (U, V, W═N).

A preferred substitution pattern on said substituent on the 1-position is R₃′, R₅′=each independently chloro, bromo, Me, OMe or OCHF₂. In one more preferred embodiment R₃′ and R₅′ are identical. In another preferred embodiment they are both chloro, both bromo, both Me, both OMe, or both OCHF₂; in another preferred embodiment R₃′ is chloro or bromo, and R₅′ is OMe. Most preferably both R₃′ and R₅′ are chloro, bromo or OCHF₂. When the 1-substituent is phenyl then R₂′ and R₆′ are preferably hydrogen. R₄′ then is preferably hydrogen, OH, chloro, bromo, Me, OMe, OCHF₂ or OX. Three most preferred substitution patterns on the phenyl as the 1-substituent are a) R₃′, R₄′, R₅′═OMe; b) R₃′=chloro, R₄′, R₅′=OMe; and c) R₄′=hydrogen, OH or OX and R₃′ and R₅′=both chloro, both bromo, or both OCHF₂. Due to the rotational freedom of the phenyl, in b) the definitions for R₃′ and R₅′ are interchangeable.

The alkyl residue in the (C₁-C₄)alkyl or (C₁-C₄)alkoxy, as used in the substituent definitions of formula (I), may be branched, unbranched or cyclic and may contain double or triple bonds. It is e.g. methyl, ethyl, n-propyl, n-butyl, isopropyl, sec-butyl, t-butyl, cyclopropyl, cyclobutyl, ethenyl, prop-2-enyl or prop-3-enyl, but-1-enyl, but-2-enyl, but-3-enyl or propargyl. Preferably it is methyl, ethyl or isopropyl; particularly preferably it is methyl.

The alkyl residue in the (C₁-C₆)alkyl or (C₁-C₆)alkoxy may be unbranched, branched or cyclic and may contain double or triple bonds. Examples of unbranched alkyls are methyl, ethyl, n-propyl, n-butyl, n-pentyl and n-hexyl. Examples of branched alkyl are isopropyl, sec-butyl, t-butyl, (1,1-diethyl)methyl, (1-propyl-1-methyl)methyl, (1-isopropyl-1-methyl)methyl, (1,1-dimethyl-1-ethyl)methyl, (1-t-butyl)methyl, (1-propyl-1-ethyl)methyl, (1-isopropyl-1-ethyl)methyl, (1,1-diethyl-1-methyl)methyl and (1-t-butyl-1-methyl)methyl. Examples of the cyclic alkyl are cyclopropyl, cyclobutyl, cyclopentyl, cyclohexyl or (2- or 3-methyl)cyclopentyl. Examples of unsaturated alkyls are ethenyl, prop-2-enyl, but-1-enyl, but-2-enyl, but-3-enyl, pent-1-enyl, pent-2-enyl, pent-3-enyl, pent-4-enyl, penta-1,3-dienyl, penta-1,4-dienyl, penta-2,4-dienyl or propargyl.

The term “halogen” means in the context of the present application fluoro, chloro or bromo.

In the context of the present invention the term “IGF-1 receptor” encompasses human IGF-1 receptor, the amino acid sequence of which is known [see e.g. T. E. Adams et al. Cellular and Molecular Life Sciences 2000, 57, p. 1050-1093], but it also encompasses other IGF-1R, such as IGF-1R of mammals in general.

The prodrug compounds of formula (II) comprise one OX group in either R₅ or R₄′, or OX groups may be present in both R₅ and R₄′ (when V designates CR₄′).

In the present invention, —OX groups designate phosphate derivatives, ester derivatives, carbonate derivatives (acyloxy derivatives of the parent compounds) and/or linked poly(ethylene glycol) derivatives as described below. Any other suitable derivatives known by those skilled in the art and considered as equivalents may also be used in the scope of the present invention.

When the active parent compounds of the present invention possess a hydroxyl group, a carbonate derivative, prepared by reacting the parent compounds with a suitable alkyl- or arylchloroformate, are exemplified as prodrugs. Particularly preferred derivatives as prodrugs are —OCOOCH₃—OCOOC₂H₅, —OCOOPropyl, —OCOOIsopropyl, —OCOOBu, —OCOO(m-COONa-Ph), —OCOOCH₂CH₂COONa, —OCOOCH₂CH₂N(CH₃)₂, and the like.

Examples of ester derivatives are formates, acetates, benzoates (e.g. OCO(m-COONa-Ph), dimethylglycine esters, aminoalkyl esters, carboxyalkyl esters, esters with amino acids and the like.

The invention also encompasses chemical modifications of the parent compounds to prolong their circulating lifetimes. Examples of suitable poly(ethylene glycol) derivatives that possess this property are described in e.g. US 2005171328 (NEKTAR THERAPEUTICS AL CORP) or U.S. Pat. No. 6,713,454 (NOBEX CORP). Since the parent compounds are lipophilic, the PEG-oligomer/polymer also increases the hydrophilicity of the prodrugs and thereby their aqueous solubility.

The selection method and the process method of an appropriate prodrug derivative are described in the literature such as Design of Prodrugs, Elsevier, Amsterdam 1985; G. R. Pettit et al. Anti-Cancer Drug Design 16 (2001) 185-193.

Most preferably, OX groups designate phosphate derivatives. Prodrug compounds of particular interest, depicted below, are (1R)-1-(3,4,5-trimethoxyphenyl)-2-formyl-5-(dihydrogen phosphate)-6-methoxy-1,2,3,4-tetrahydroisoquinoline, (1R)-1-(3,5-dichlorophenyl)-2-formyl-5-(dihydrogen phosphate)-6-difluoromethoxy-1,2,3,4-tetrahydroisoquinoline and (1R)-1-[3,5-dichloro-4-(dihydrogen phosphate)phenyl]-2-formyl-6-difluoromethoxy-1,2,3,4-tetrahydroisoquinoline and their corresponding 6-(2,2,2-trifluoroethoxy), 2-cyano and 2-acetyl derivatives, and salts thereof.

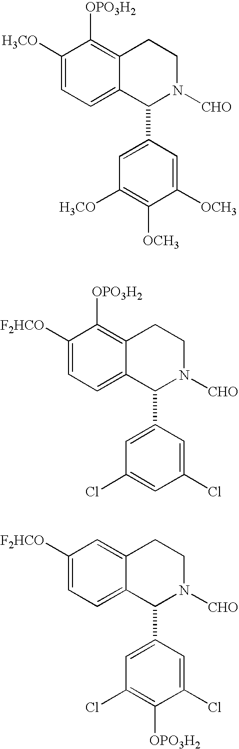

The above substances may be synthesized from their parent compounds, which contain a 5- or a 4′-hydroxy group, by reaction with phosphoroxychloride, followed by hydrolysis to the corresponding phosphate [see e.g. U.S. Pat. No. 5,637,680 (Etoposide phosphate)]. Other suitable reagents are dibenzyl phosphite in combination with carbon tetrachloride (Example 1) and diethyl chlorophosphate (Examples 4 and 6).

The solubility of the prodrug compound 1-(3,4,5-trimetoxyphenyl)-2-formyl-5-(dihydrogen phosphate)-6-methoxy-1,2,3,4-tetrahydroisoquinoline in a sodium phosphate buffer at pH 7.4 was found to be in excess of 50 milligram/ml compared to the solubility of the corresponding parent compound that is about 20 microgram/ml (example 2).

According to the present invention, pharmaceutically acceptable salts are produced from acidic inorganic or organic compounds, or alkaline inorganic or organic compounds.

As used herein, the phrase “pharmaceutically acceptable salt” refers to a salt that retains the biological effectiveness of the free acids and bases of a specified compound and that is not biologically or otherwise undesirable. The pharmaceutically acceptable salts of the compounds of formula (I) and/or the prodrug compounds of formula (II) are acid addition salts with pharmaceutically acceptable acids, which are possible in the case where R₂ is hydrogen, Me or Et; and/or at least one of U, V and W is nitrogen, and when the group OX contains a basic nitrogen atom.

A desired salt may be prepared by any suitable method known in the art, including treatment of the free base with an inorganic acid, such as hydrochloric acid, hydrobromic acid, sulphuric acid, nitric acid, phosphoric acid, and the like, or with an organic acid, such as formic acid, acetic acid, maleic acid, succinic acid, mandelic acid, maleic acid, fumaric acid, malonic acid, pyruvic acid, oxalic acid, glycolic acid, salicylic acid; a pyranosidyl acid, such as glucuronic acid or galacturonic acid; an alpha-hydroxy acid, such as citric acid or tartaric acid; an amino acid, such as aspartic acid or glutamic acid; an aromatic acid, such as benzoic acid or cinnamic acid; a sulfonic acid, such as methanesulfonic acid, p-toluenesulfonic acid or ethanesulfonic acid; or the like.

In the present invention the preferred ammonium salts are derived from hydrochloric, hydrobromic, methanesulfonic, acetic, propionic, benzoic, citric, tartaric, malic, maleic, fumaric, lactic, nitric, and phosphoric or succinic acid.

Generally, the salts are prepared by reacting the free base with stoichiometric amounts or with an excess of the desired salt forming inorganic or organic acid in a suitable solvent or various combinations of solvents. For example, the free base can be dissolved in a mixed aqueous solution of the appropriate acid and the salt recovered by standard techniques, for example, by evaporation of the solution. Alternatively, the free base can be charged into an organic solvent such as a lower alkanol, symmetrical or asymmetrical ethers containing 2 to 10 carbon atoms, an alkyl ester, or mixtures thereof, and the like, and then it is treated with the appropriate acid to form the corresponding salt. The salt is recovered by standard recovery techniques, for example, by filtration of the desired salt from the mixture, or it can be precipitated by the addition of a solvent in which the salt is insoluble and recovered there from.

Examples of suitable inorganic and organic solvents for performing the various reactions include any inorganic or organic solvent that does not adversely affect the reactants or the resulting product, including halogenated solvents such as methylene chloride, chloroform, ether solvents such as diethyl ether, and other solvents such as tetrahydrofuran, dioxane, diglyme, cyclooctane, benzene or toluene, heptane, cyclohexane, aliphatic as well as cycloaliphatic and aromatic hydrocarbon solvents, water, acidified aqueous solutions, mixed organic and inorganic solutions, ethyl acetate, propyl acetate and mixtures thereof.

Also encompassed by the present invention are salts formed from acidic prodrugs, such as phosphates, and alkaline inorganic or organic compounds. Preferred inorganic cations comprised in the salts are lithium, sodium, potassium, rubidium, ammonium, calcium, magnesium, zinc and manganese. Production of phosphate salts are described in e.g. G. R. Pettit et al. Anti-Cancer Drug Design 16 (2001) 185-193.

Preferred salts also include those formed from acidic prodrugs and organic amines, including, but not limited to, imidazole and morpholine. Alkaline amino acid salts may also be used. The term “amino acids” designates, according to the invention, in particular the [alpha]-amino acids occurring in nature, but moreover also includes their homologues, isomers and derivatives. Enantiomers can be mentioned as an example of isomers. Derivatives can be, for example, amino acids provided with protective groups. Preferred alkaline amino acid are arginine, ornithine, diaminobutyric acid, lysine or hydroxy lysine and especially L-arginine, L-lysine or L-hydroxy lysine; an alkaline dipeptide or a pharmaceutically acceptable alkaline amino acid derivate.

The compounds of formula (I) according to the invention can be prepared using the general methods described in PCT/CH2004/000147, the content of which is incorporated herein by reference in its entirety. It is understood that any other suitable methods known to the skilled in the art may also be encompassed by the scope of the present invention.

In PCT/CH2004/000147, one starting material is a phenethylamine substituted in the aromatic part. In the present invention, the starting material is a phenethylamine that in addition may have a substituent on the benzylic carbon atom. Some of these starting materials are available by alkylation of a suitable substituted phenylacetonitril, followed by reduction to the amine [Organic Syntheses, Coll. Vol. 76, p. 169; Sukata, K. Bull. Chem. Soc. Jpn. 56 (1983) 3306-3307], or via substituted β-nitrostyrenes [Ambros, R. et al. J. Med. Chem. 33 (1990) 153-160; Schäfer, H. et al. Tetrahedron 51 (1995) 2305-2334].

It will be appreciated by those skilled in the art that in processes of the present invention certain functional groups such as hydroxyl groups in the starting reagents or intermediate compounds may need to be protected by protecting groups. Thus, the preparation of the compounds (I) may involve the addition and removal of one or more protecting groups. The protection and deprotection of functional groups is described in “Protective Groups in Organic Chemistry”, edited by J. W. F. McOmie, Plenum Press (1973) and “Protective Groups in Organic Synthesis”, 2^{nd} edition, T. W. Greene and P. G. M. Wuts, Wiley-Interscience (1991) and “Protecting Groups” 3^{rd} edition, P. J. Kocienski, Georg Thieme Verlag (2005).

Suitable protecting groups for aromatic hydroxyl groups in the present invention are e.g. benzyl and isopropyl groups. Removal of the benzyl group and the isopropyl group is easily achieved by catalytic hydrogenation (catalyst Pd/carbon) and treatment with BCl₃, respectively. Another useful reagent is trimethyliodosilane, which selectively removes isopropyl groups in the presence of difluoromethoxy groups.

The parent compounds of prodrugs (II) resulting from the biotransformation of the prodrug compounds according to the invention can be prepared using the methods described in the following Examples 1, 4, 5 and 6 as well as in PCT/CH2004/000147, the content of which is incorporated herein by reference in its entirety. Some preferred compounds (I) contain one or several difluoromethoxy groups. An intermediate in the synthesis of some compounds (I) is 2-(3-difluoromethoxyphenyl)ethylamine, which is synthesized from 3-difluoromethoxybenzaldehyde (commercially available) according to the general procedures described in PCT/CH2004/000147. Another useful starting material is 2-benzyloxy-3-difluoromethoxybenzaldehyde, which is available by difluoromethylation [in analogy with Guay, D. et al. Med. Chem. Lett. 12 (2002) 1457-1-461] of 2-benzyloxy-3-hydroxybenzaldehyde [Kessar, S. V. et al. J. Org. Chem. 53 (1988) 1708-1713]. Other starting materials can be produced from suitable hydroxylated benzoic acids. One such example is 3,5-dihydroxybenzoic acid, which upon treatment with methyl chlorodifluoroacetate (or chlorodifluaoromethane) and potassium carbonate in dimethylformamide followed by hydrolysis gives 3,5-di(difluoromethoxy)benzoic acid. It is understood that any other suitable methods known to the skilled in the art may also be encompassed by the scope of the present invention.

The compounds and the prodrug compounds of the present invention contain at least one chiral centre and therefore may exist in different enantiomeric forms. Although particularly preferred compounds (I) and prodrug compounds (II) are enantiomerically pure the scope of the present invention is intended to cover both enantiomers per se, as well as mixtures of them in any ratio, such as racemic mixtures.

Prodrug compounds (II) of the present invention may be obtained in their enantiomerically pure forms by using enantiomerically pure parent compounds as starting material. Enantiomerically pure compounds (I) and prodrug compounds (II) may also be obtained from their racemates by crystallization of their addition salts with chiral acids [see e.g. D. L. Minor et al. J. Med. Chem. 37 (1994) 4317-4328; U.S. Pat. No. 4,349,472], or alternatively, may be isolated by preparative HPLC using commercially available chiral phases. Other routes to the pure enantiomers of compounds (I) and of the parent compounds of prodrugs (II) of the present invention are the use of asymmetric synthesis [M. J. Munchhof et al. J. Org. Chem. 60 (1995) 7086-7087; R. P. Polniaszek et al. Tetrahedron Letters 28 (1987) 4511-4514], by asymmetric transfer hydrogenation of the intermediate imines (II) or iminium salts (III) [N. Uematsu et al. J. Am. Chem. Soc. 118 (1996) 4916-4917; G. Meuzelaar et al. Eur. J. Org. Chem. 1999, 2315-2321], or by resolution of chiral diastereometric derivatives thereof, as known by those skilled in the art.

The compounds (I) and/or prodrug compounds of formula (II) and their pharmaceutically acceptable salts, where applicable, may be administered in the form of a pharmaceutical composition in which they are in association with a pharmaceutically acceptable adjuvant, diluent or carrier, in order to prevent or treat any disease in which inhibition of the IGF-1 receptor would be considered beneficial by the skilled person. The present invention also provides a pharmaceutical composition comprising the prodrug compounds of formula (I), or a pharmaceutically acceptable salt thereof, as hereinbefore defined, in association with a pharmaceutically acceptable adjuvant, diluent or carrier. As to the appropriate excipients, diluents and adjuvant, reference may be made to the standard literature describing these, e.g. to chapter 25.2 of Vol. 5 of “Comprehensive Medicinal Chemistry”, Pergamon Press 1990, and to “Lexikon der Hilfsstoffe für Pharmazie, Kosmetik und angrenzende Gebiete”, by H. P. Fiedler, Editio Cantor, 2002 (in German).

The compounds (I) and/or prodrug compounds of formula (II) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi permeable matrices of solid hydrophobic polymers containing the prodrug compounds (I), which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and [gamma]ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The pharmaceutical compositions of the invention will preferably comprise from 0.001 to 50% by weight of compound (I).

The prodrug compounds (II) once transformed by the organism into their corresponding active parent compounds have IC₅₀ activities in intact cell systems ranging from 8 microgram/ml to 150 picogram/ml. Due to the large difference in activities, the pharmaceutical compositions of the invention will preferably comprise from 0.001 to 50% by weight of prodrug compounds (II).

The daily dose of the compounds (I) and/or prodrug compounds of formula (II) will necessarily be varied depending upon the host treated, the particular route of administration, and the severity and kind of the illness being treated. Accordingly the optimum dosage may be determined by the practitioner who is treating any particular patient.

The pharmaceutical compositions of the invention may be formulated as creams, gels, solutions, ointments, suspensions or plasters etc. when intended for topical administration; for administration by inhalation, e.g. as aerosols or dry powders; for oral administration, e.g. in the form of tablets, capsules, gels, syrups, suspensions, solutions, powders or granules; for rectal or vaginal administration e.g. as suppositories; or for parenteral injection (including intravenous, subcutaneous, intramuscular, intravascular, or infusion) as a sterile solution, suspension or emulsion.

The compounds (I) and/or the prodrug compounds (II) of the present invention once transformed by the organism into corresponding active parent compounds were found to down-regulate or inhibit the expression or function of the human IGF-1 receptor, without inhibiting the structurally closely related insulin receptor. They were found to promote apoptosis of malignant cells and to interfere with cell division by blocking the cells in the prophase of the mitotic cycle. The resulting active parent compounds are useful for the prevention and/or treatment of diseases of unregulated IGF-1R expression, including cell proliferate diseases such as cancer, atherosclerosis, restenosis, inflammatory diseases e.g. psoriasis, autoimmune diseases e.g. rheumatoid arthritis, and transplant rejection.

“Treatment” refers to both therapeutic treatment and prophylactic or preventative measures. Those in need of treatment include those already with the disorder as well as those in which the disorder is to be prevented. Hence, the mammal to be treated herein may have been diagnosed as having the disorder or may be predisposed or susceptible to the disorder.

“Mammal.” for purposes of treatment refers to any animal classified as a mammal, including, but not limited to, humans, domestic and farm animals or pet animals, such as dogs, horses, cats, cows, monkeys etc. Preferably, the mammal is human.

The term “therapeutically effective amount” refers to an amount of a drug effective to treat a disease or disorder in a mammal. In the case of cancer, the therapeutically effective amount of the drug may reduce the number of cancer cells; reduce the tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; and/or relieve to some extent one or more of the symptoms associated with the cancer. To the extent the drug may prevent growth and/or kill existing cancer cells, it may be cytostatic and/or cytotoxic. The phrase “therapeutically effective amount” is used herein to mean an amount sufficient to prevent, or preferably reduce by at least about 30 percent, preferably by at least 50 percent, preferably by at least 70 percent, preferably by at least 80 percent, preferably by at least 90%, a clinically significant change in the growth or progression or mitotic activity of a target cellular mass, group of cancer cells or tumour, or other feature of pathology.

The terms “cancer” and “cancerous” refer to or describe the physiological condition in mammals that is typically characterized by unregulated cell growth.

Some examples of cancers in which IGF-1R is unregulated or over expressed and which can be prevented and/or treated by the resulting active parent compounds include, but are not limited to, cancer of the breast, prostate, colon, lung, brain, kidney, pancreas, and melanoma, multiple myeloma, lymphoma and leukemia.

Optionally the compounds (I) and/or prodrug compounds of formula (II) may be used against cell proliferate diseases in combination with conventional treatments such as irradiation and/or one or more chemotherapeutic agents such as e.g. Actinomycin, Altretamine, Bleomycin, Busulphan, Capecitabine, Carboplatin, Carmustine, Chlorambucil, Cisplatin, Cladribine, Crisantaspase, Cyclophosphamid, Cytarabine, Dacarbazine, Daunorubicin, Doxorubicin, Epirubicin, Etoposide, Fludarabine, Eluorouracil, Gemcitabine, Idarubicin, Ifosfamide, Irinotecan, Lomustine, Melphalan, Mercaptopurine, Methotrexate, Mitomycin, Mitoxantrone, Oxaliplati, Pentostatin, Procarbazine, Streptozocin, Taxol, Temozolomide, Thiotepa, Tioguanine/Thioguanine, Topotecan, Treosulfan, Vinblastine, Vincristine, Vindesine or Vinorelbine.

When a chemotherapeutic agent is used in combination with the compounds (I) and/or prodrug compounds of formula (II), then this may be used in the form of a medicament containing a combination of these two agents, for simultaneous administration, or they may be used in the form of separate dosage forms, each containing one of the agents, and in the latter case the individual dosage forms may be used e.g. sequentially, i.e. one dosage form with the compounds (I) and/or prodrug compounds of formula (II), followed by a dosage form containing the chemotherapeutic agent (or vice versa). This embodiment of two separate dosage forms may be conceived and provided in the form of a kit.

Generally, the Kit comprises a container and a label or package insert on or associated with the container. Suitable containers include, for example, bottles, vials, syringes, etc. The containers may be formed from a variety of materials such as glass or plastic. The container holds a prodrug composition or the transformed active parent composition that is effective for treating the condition and may have a sterile access port (for example the container may be an intravenous solution bag or a vial having a stopper pierceable by a hypodermic injection needle). The label or package insert indicates that the composition is used for treating the condition of choice, such as cancer.

In addition to their use in therapeutic medicine, the compounds (I) and/or prodrug compounds of formula (II) and their pharmaceutically acceptable salts are also useful as pharmacological tools in the development and standardization of in vitro and in vivo test systems for the evaluation of the effects of inhibitors of cell cycle activity in laboratory animals such as cats, dogs, rabbits, monkeys, rats and mice, as part of the search for new therapeutic agents.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

The foregoing description will be more fully understood with reference to the following Examples. Such Examples, are, however, exemplary of methods of practicing the present invention and are not intended to limit the scope of the invention.

### EXAMPLES

Products described in the Examples have satisfactory proton nuclear magnetic resonance spectra and/or mass spectral data. Melting points are uncorrected.

### Example 1

### (1R)-1-(3,4,5-trimethoxyphenyl)-2-formyl-5-(dihydrogen phosphate)-6-methoxy-1,2,3,4-tetrahydroisoquinoline

1. 2-Benzyloxy-3-methoxyphenylethylamine (74.7 g) was added to an aqueous solution of sodium hydroxide (450 ml, 2M) and dichloromethane (300 mL). To the vigorously stirred mixture containing the amine, 3,4,5-trimethoxybenzoyl chloride (66.8 g) dissolved in dichloromethane (250 ml) was added during 30 minutes at room temperature. After the addition, the mixture was stirred for further 60 minutes. The dichloromethane phase was separated, washed with hydrochloric acid (200 ml, 2M), dried (sodium sulphate) and concentrated to dryness. The residual amide (137.4 g) was used without further purification for the production of the corresponding imine.

2. A mixture of the amide from step 1 (105.0 g), toluene (500 ml) and phosphorus oxychloride (160 ml) was heated under reflux for 2.0 hours. The reaction mixture was cooled down to room temperature and filtered. The retained crystals were washed with toluene (200 ml) followed by diethyl ether (200 ml) giving the expected imine hydrochloride (79.8 g). An analytical sample was obtained by crystallization from methanol-diethyl ether giving a white solid, m.p. 200-204° C.°.

3. The imine (47.0 g) produced according to step 2 was dissolved in a mixture of methanol (250 ml) and 1,2-dimethoxyethane (250 ml) and treated with sodium borohydride at 10° C. until no starting material remained (TLC: silica gel/ethyl acetate). The mixture was concentrated to dryness and partitioned between aqueous sodium hydroxide (400 ml, 2M) and dichloromethane (400 ml). The organic phase was separated, dried and concentrated to dryness, leaving the secondary amine (46.4 g). An analytical sample was obtained by crystallization from ethanol, giving a white solid, m.p. 122-124° C.

4. The secondary amine (40.0 g) produced according to method A was dissolved in hot ethanol (1000 ml) and the solution was mixed with acetyl-D-leucine (16.0 g) dissolved in hot ethanol (400 ml). The mixture was stirred and filtered when the slurry had reached 45° C. The retained crystals were washed with ethanol (1000 ml) and dried giving a white solid (24.8, 53.1% ee). A second crystallization (24.5 g) from ethanol (950 ml) gave a white solid (13.9 g, >99.9% ee), m.p. 209-212° C., [α]_{D}²⁰-53.5° (c=1.0, DMF).

5. The acetyl-D-leucine salt (29.5 g) produced according to step 1 above was partitioned between dichloromethane (300 ml) and aqueous sodium hydroxide (200 ml, 2M). The organic phase was dried and concentrated to dryness leaving the secondary amine. A solution of the amine, toluene (400 ml) and formic acid (20 ml) was refluxed for 18 hours using a Dean-Stark trap. The reaction mixture was concentrated to dryness, leaving the formyl derivative of the 5-benzylether as a viscous oil. A solution of the residue in a mixture of dimethylformamide (200 ml) and ethanol (100 ml) was reacted with hydrogen in the presence of palladium on carbon (2.5 g, 5%) for two hours. The mixture was filtered and the filtrate concentrated to dryness. The residue was crystallized from ethanol leaving (1R)-1-(3,4,5-trimethoxyphenyl)-2-formyl-5-hydroxy-6-methoxy-1,2,3,4-tetrahydroisoquinoline (20.1 g), m.p. 190-192° C., [α]_{D}²⁰-191.2° (c=1.0, CHCl₃).

6. A solution of (1R)-1-(3,4,5-trimethoxyphenyl)-2-formyl-5-hydroxy-6-methoxy-1,2,3,4-tetrahydroisoquinoline (11.0 g), carbon tetrachloride (25 ml.), diisopropylethylamine (17 ml) and 4-dimethylaminopyridine (400 mg) in a mixture of acetonitrile (100 ml) and dimethylformamide (40 ml) was cooled to −10° C. Dibenzylphosphite (25 g, 80% purity) was added drop-wise at −5° C. to −10° C. with stirring. After stirring for 5 hours at −5° C., the reaction was terminated by the drop-wise addition of an aqueous solution of potassium dihydrogen phosphate (50 ml., 0.5 M) followed by water (400 ml). The mixture was extracted with ethyl acetate (2×300 ml), and the organic phase was dried and concentrated to dryness. The residue was purified by chromatography on silica gel (250×6 cm) using ethyl acetate as eluent. The second fraction contained a small amount of the starting material and predominately its 5-O-dibenzylphosphoryl derivative, which was obtained as a viscous oil (15.0 g).

7. A solution of (1R)-1-(3,4,5-trimethoxyphenyl)-2-formyl-5-(dibenzyl phosphate)-6-methoxy-1,2,3,4-tetrahydroisoquinoline (15.0 g), produced according to step 6 above, in ethanol (200 ml) was stirred with palladium on carbon (2.5 g, 5%) in a hydrogen atmosphere for 2 hours. The slurry was filtered and the filtrate concentrated to dryness. The residue (10.8 g) was partitioned between water (400 ml) and dichloromethane (2×100 ml). The water phase was concentrated to dryness, and the residue (8.6 g) was crystallized from 2-propanol giving (1R)-1-(3,4,5-trimethoxyphenyl)-2-formyl-5-(dihydrogen phosphate)-6-methoxy-1,2,3,4-tetrahydroisoquinoline (7.2 g), m.p. 138-141° C., [α]_{D}²⁰−145.2° (c=1.0, methanol).

### Example 2

### Solubility of (1R)-1-(3,4,5-trimethoxyphenyl)-2-formyl-5-(dihydrogen phosphate)-6-methoxy-1,2,3,4-tetrahydroisoquinoline in a Physiologically Acceptable Buffer

The solubility of the title compound in a sodium phosphate buffer at pH 7.4 was found to be in excess of 50 milligram/ml. The corresponding solubility of the parent compound is about 20 microgram.

### Example 3

### Dephosphorylation of (1R)-1-(3,4,5-trimethoxyphenyl)-2-formyl-5-(dihydrogen phosphate)-6-methoxy-1,2,3,4-tetrahydroisoquinoline by Alkaline Phosphatase

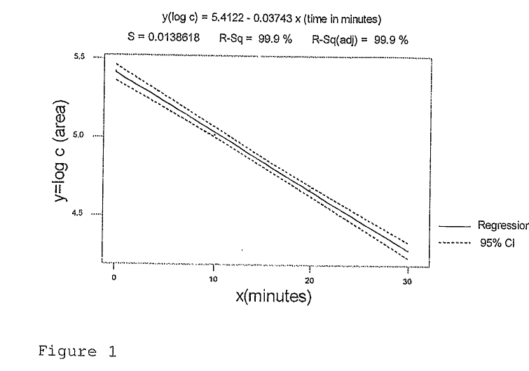

The ability of bovine alkaline phosphatase (type VII S, Sigma-Aldrich) to dephosphorylate the title compound was investigated in vitro. It was found that the half-life time is 8.0 minutes at a phosphatase concentration of 10 units/ml (37° C., pH 7.4). Intravenous administration of the title compound is consequently expected to result in a rapid formation of the active moiety (1R)-1-(3,4,5-trimethoxyphenyl)-2-formyl-5-hydroxy-6-methoxy-1,2,3,4-tetrahydroisoquinoline (see FIG. 1).

### Example 4

### (1R)-1-(3,5-dichlorophenyl)-2-acetyl-5-(dihydrogen phosphate)-6-ethoxy-1,2,3,4-tetrahydroisoquinoline

1. (1R)-1-(3,5-dichlorophenyl)-2-acetyl-5-hydroxy-6-ethoxy-1,2,3,4-tetrahydroisoquinoline [(6.2 g), m.p. 238-241° C., [α]_{D}²⁰-128.9° (c=1.0, CHCl₃)], produced in analogy with example 3 above, was dissolved in ethanol free chloroform (150 ml). To the solution were added diisopropylethylamine (15 ml) and 4-dimethylaminopyridine (200 mg) followed by the drop-wise addition of diethylchlorophosphate (6 ml). After stirring for 5 hours, water (200 ml) was added. The organic phase was separated, washed with hydrochloric acid (0.5 M, 200 ml), dried and concentrated to dryness. The residue was purified by chromatography on silica gel (250×6 cm) using ethyl acetate as eluent, giving the 5-O-diethylphosphoryl derivative as a solid (7.9 g).

2. Trimethylbromosilane (9.2 g) was added to a solution of (1R)-1-(3,5-dichlorophenyl)-2-acetyl-5-(diethyl phosphate)-6-ethoxy-1,2,3,4-tetrahydroisoquinoline (7.5 g) in dichloromethane (50 ml). After stirring at room temperature for 17 hours, dichloromethane and (150 ml) and water (200 ml) were added. The organic phase was separated, dried and concentrated to dryness, leaving a white solid (6.2 g). Crystallization from ethanol-water gave (1R)-1-(3,5-dichlorophenyl)-2-acetyl-5-(dihydrogen phosphate)-6-ethoxy-1,2,3,4-tetrahydroisoquinoline (5.3 g) as a white solid, m.p. 225-228° C., [α]_{D}²⁰-79.7° (c=1.0, methanol).

### Example 5

### 1-(3,5-dichloro-4-hydroxyphenyl)-2-formyl-6-difluoromethoxy-1,2,3,4-tetrahydroisoquinoline

1. A mixture of ethyl 3,5-dichloro-4-hydroxybenzoate (100.0 g), potassium carbonate (60 g) and benzyl chloride (98 ml) in dimethylformamide (400 ml) was heated at 55° C. for 15 hours. The slurry was filtered and the filtrate concentrated to dryness. The residue was crystallized from methanol, giving ethyl 4-benzyloxy-3,5-dichlorobenzoate (104.2 g) as a white solid, m.p. 66-68° C.

2. A mixture of ethyl 4-benzyloxy-3,5-dichlorobenzoate (103.0 g), potassium hydroxide (32 g) and ethanol-water (1000 ml, 8:2) was stirred at room temperature for 3 hours. The solution was concentrated to dryness, and the residue was partitioned between aqueous hydrochloric acid (1 M, 500 ml) and chloroform-ethanol (1000 ml, 3:2). The organic phase was dried and concentrated to dryness leaving 4-benzyloxy-3,5-dichlorobenzoic acid (90.2 g). An analytical sample was obtained by crystallization from ethanol, giving 4-benzyloxy-3,5-dichlorobenzoic acid as a white solid, m.p. 211-213° C.

3. A solution of 4-benzyloxy-3,5-dichlorobenzoic acid (64.8 g) in dimethylformamide (500 ml) was treated with 1,1′-carbonyldiimidazole (40.5 g) at 50° C. for one hour. The solution was cooled to 25° C. and a solution of 2-(3-difluoromethoxyphenyl)ethylamine (40.8 g) in dimethylformamide (150 ml) was added dropwise. After stirring for one hour, the mixture was partitioned between water (1000 ml) and t-butyl methyl ether (500 ml). The organic phase was washed with aqueous hydrochloric acid (1M, 300 ml) followed by aqueous sodium hydroxide (1M, 200 ml), dried and concentrated to dryness. The residue was purified by chromatography on silica gel (8×40 cm) using dichloromethane-ethyl acetate (9:1) as eluent, giving the pure amide (84.0 g). An analytical sample was obtained by crystallization from methanol, m.p. 99-100° C.

4. A mixture of the amide from step 3 (4.0 g), xylene (50 ml) and phosphorus oxychloride (1.5 ml) was heated under reflux for 67 hours. The reaction mixture was concentrated to dryness and the residue was partitioned between dichloromethane (200 ml) and aqueous sodium hydroxide (2M, 200 ml). The organic phase was dried and concentrated to dryness. The residue was purified by chromatography on silica gel (6×25 cm) using a mixture of dichloromethane and ethyl acetate (97:3) as eluent, giving the imine (0.8 g) as viscous oil.

5. The imine (0.8 g) produced according to step 4 was dissolved in methanol and (100 ml) treated with sodium borohydride at room temperature until no starting material remained (TLC: silica gel/ethyl acetate). The mixture was concentrated to dryness and partitioned between aqueous sodium hydroxide (2M, 200 ml) and dichloromethane (200 ml). The organic phase was separated, dried and concentrated to dryness, leaving the crude secondary amine (0.75 g) as viscous oil.

6. A solution of the amine (0.75 g), toluene (100 ml) and formic acid (2 ml) was refluxed for 3 hours using a Dean-Stark trap. The reaction mixture was concentrated to dryness, leaving the formyl derivative as a gum.

7. A solution of the formyl derivative from step 6 (300 mg) in ethyl acetate (50 ml) containing two drops of concentrated hydrochloric acid was reacted with hydrogen in the presence of palladium on carbon (100 mg, 10%) for two hours. The mixture was filtered and the filtrate concentrated to dryness leaving 1-(3,5-dichloro-4-hydroxyphenyl)-2-formyl-6-difluoromethoxy-1,2,3,4-tetrahydroisoquinoline as a gum, which solidified by treatment with diethyl ether, m.p. 173-1760° C.

### Example 6

### (1R)-1-[3,5-dichloro-4-(dihydrogen phosphate)phenyl]-2-formyl-6-(2,2,2-trifluoroethoxy)-1,2,3,4-tetrahydroisoquinoline

1. A mixture of ethyl 3,5-dichloro-4-hydroxybenzoate (102.7 g), potassium carbonate (60 g) and isopropyl bromide (80 ml) in dimethylformamide (600 ml) was heated at 55° C. for 15 hours. The slurry was filtered and the filtrate concentrated to dryness. The residue was partitioned between t-butyl methyl ether (700 ml) and an aqueous sodium hydroxide solution (2M, 400 ml). The organic phase was dried and concentrated to dryness giving ethyl 4-isopropoxy-3,5-dichlorobenzoate (109.0 g) as viscous oil.

2. A mixture of ethyl 3,5-dichloro-4-isopropoxy-benzoate (103.0 g), potassium hydroxide (32 g) and ethanol-water (800 ml, 8:2) was stirred at room temperature for 3 hours. The solution was concentrated to dryness, and the residue was partitioned between aqueous hydrochloric acid (1 M, 500 ml) and chloroform-ethanol (1000 ml, 3:2). The organic phase was dried and concentrated to dryness leaving 4-isopropoxy-3,5-dichlorobenzoic acid (90.2 g). The residue was crystallized from ethanol-water (1:1) giving 1-isopropoxy-3,5-dichlorobenzoic acid as a white solid (79.2 g), m.p. 140-1.42° C.

3. A solution of 3,5-dichloro-4-isopropoxybenzoic acid (61.5 g) in dimethylformamide (500 ml) was treated with 1,1′-carbonyldimidazole (44.4 g) at 50° C. for one hour. The solution was cooled to 25° C. and a solution of 2-(3-benzyloxyphenyl)ethylamine (56.0 g) in dimethylformamide (150 ml) was added dropwise. After stirring for one hour, the mixture was partitioned between water (1000 ml) and t-butyl methyl ether (500 ml). The organic phase was washed with aqueous hydrochloric acid (1M, 300 ml) followed by aqueous sodium hydroxide (1M, 200 ml), dried and concentrated to dryness leaving the crude amide (109.7 g) as viscous oil.

4. A mixture of the amide from step 3 (109.7 g), toluene (550 ml) and phosphorus oxychloride (180 ml) was heated under reflux for 2.5 hours. The reaction mixture was concentrated to dryness and the residue was partitioned between dichloromethane (1000 ml) and aqueous sodium hydroxide (2M, 600 ml). The organic phase was dried and concentrated to dryness giving the crude imine (110 g).

5. The imine (110 g) produced according to step 4 was dissolved in a mixture of methanol and (500 ml) and tetrahydrofuran (500 ml) and treated with sodium borohydride at room temperature until no starting material remained (TLC: silica gel/ethyl acetate). The mixture was concentrated to dryness and partitioned between aqueous sodium hydroxide (2M, 400 ml) and dichloromethane (600 ml). The organic phase was separated, dried and concentrated to dryness, leaving the crude secondary amine (109 g) as viscous oil. The amine was converted into its hydrochloride, which was isolated as a white crystalline powder (59.2 g). An analytical sample was obtained crystallization from methanol, m.p. 220-240° C. (dec.).

6. The secondary amine (24.5 g, generated from the hydrochloride), produced according to step 5, was dissolved in hot ethanol (400 ml) and the solution was mixed with acetyl-D-leucine (10.0 g) dissolved in hot ethanol (100 ml). The mixture was allowed to stand at room temperature for 20 hours, after which it was filtered. The retained crystals were washed with ethanol (200 ml) and dried giving a white solid (15.6 g, 79.5% ee). A second crystallization (15.2 g) from ethanol (370 ml) gave a white solid (13.1 g, 99.0% ee), m.p. 188-192° C., [α]_{D}²⁰-21.2° (c=1.0, DMF).

7. The acetyl-D-leucine salt (12.4 g), produced according to step 6 above, was partitioned between dichloromethane (300 ml) and aqueous sodium hydroxide (2M, 200 ml). The organic phase was dried and concentrated to dryness, leaving the secondary amine. A solution of the amine, toluene (200 ml) and formic acid (20 ml) was refluxed for 18 hours using a Dean-Stark trap. The reaction mixture was concentrated to dryness, leaving the formyl derivative as a gum. A solution of the residue in ethyl acetate (130 ml) containing 0.3 ml of concentrated hydrochloric acid was reacted with hydrogen in the presence of palladium on carbon (0.5 g, 5%) for two hours. The mixture was filtered and the filtrate concentrated to dryness. The residue was crystallized from methanol leaving (1R)-1-(3,5-dichloro-4-isopopropoxyphenyl)-2-formyl-6-hydroxy-1,2,3,4-tetrahydroisoquinoline (4.2 g), m.p. 196-198° C., [α]_{D}²⁰-207.7° (c=0.4, CHCl₃).

8. A solution of (1R)-1-(3,5-dichloro-4-isopopropoxyphenyl)-2-formyl-6-hydroxy-1,2,3,4-tetrahydroisoquinoline (7.0 g) in dimethylformamide (70 ml) was treated with lithium hydride (350 mg) at 80° C. for 40 minutes. 2,2,2-Trifluoroethyl methansulphonate (5.9 g) was added and the solution was heated at 120° C. for 20 hours, after which the mixture was partitioned between aqueous hydrochloric acid (2M, 300 ml) and dichloromethane (400 ml). The organic phase was washed with aqueous sodium hydroxide (2M, 300 ml), dried and concentrated to dryness. The residue was purified by chromatography on silica gel using dichloromethane-ethylacetate (9:1) as eluent, giving (1R)-1-(3,5-dichloro-4-isopopropoxyphenyl)-2-formyl-6-(2,2,2-trifluoroethoxy)-1,2,3,4-tetrahydroisoquinoline (4.0 g) as a viscous oil, [α]_{D}²⁰-500 (c=1.1, CHCl₃).

9. A solution of (1R)-1-(3,5-dichloro-4-isopropoxyphenyl)-2-formyl-6-(2,2,2-trifluoroethoxy)-1,2,3,4-tetrahydroisoquinoline (3.8 g) in dichloromethane (40 ml) was treated with a solution of boron trichloride in dichloromethane(1M, 24 ml) at −20° C. for 10 minutes. The reaction mixture was kept at room temperature for 30 minutes, after which dichloromethane (200 ml) and water (200 ml) were added. The mixture was vigorously mixed for 20 minutes, after which the organic phase was separated, dried and concentrated to dryness leaving (1R)-1-(3,5-dichloro-4-hydroxyphenyl)-2-formyl-6-(2,2,2-trifluoroethoxy)-1,2,3,4-tetrahydroisoquinoline (3.5 g) as an amorphous solid, [α]_{D}²⁰-55° (c=1.0, CHCl₃).

10. The 4′-O-diethylphosphoryl derivative of the substance described in step 9 above was synthesized as outlined in Example 4, step 1. The product was isolated as viscous oil.

11. The Title substance was obtained by treatment of the 4′-O-diethylphosphoryl derivative (step 10) with trimethylbromosilane basically as described in Example 4, step 2. The product was obtained as an amorphous solid, [α]_{D}²⁰-38.9° (c=1.15, methanol).

### Example 7

### Inhibition of the Phosphorylation of MAPK and AKT in DU-145 Cells by (1R)-1-(3,4,5-trimethoxyphenyl)-2-formyl-5-hydroxy-6-methoxy-1,2,3,4-tetrahydroisoquinoline

DU-145 cells (prostate cancer) were incubated over night with the title compound in serum free medium. After stimulation for 15 minutes with IGF-1 (50 nM), the cells were lyzed and the lysates analysed by immunoblotting for phospho-MAPK and phospho-AKT. It was found that the phosphorylation of MAPK (Erk1/2) as well as AKT were inhibited by the presence of the title compound in a dose dependant manner with an IC₅₀ of 50 nM and 40 nM, respectively. Picropodophyllin, used as a standard, showed an IC₅₀ of around 5 μM for the inhibition of phospho-MAPK (Erk1/2).

1. A compound of the following general formula (I): 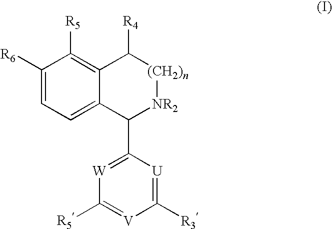
wherein
R₄ designates H; OH; CN; trifluoromethyl; NH₂; NHCN; NHCOCH₃; NHCOCH₂CH₃; NHCHO; NHCOOCH₃; amino(C₁-C₆)alkyl; amino(C₁-C₃)dialkyl; (C₁-C₆)alkoxy; (C₁-C₆)alkyl; carbonyl-R₉ wherein R₉ designates hydrogen, (C₁-C₆)alkyl, (C₁-C₆)alkoxy; (C₁-C₆)alkyl-R₁₀; (C₁-C₆)alkoxy-R₁₀; amino(C₁-C₆)alkyl-R₁₀ and amino(C₁-C₃)dialkyl-R₁₀ whereby R₁₀ designates at least one OMe, OEt, OPr, OIsopropyl, OH, CN, NH₂, ester groups with (C₁-C₃)alkyl, carbonate groups with (C₁-C₃)alkyl;
R₂ designates hydrogen, Me, Et, CHO, CN, OH, OMe, COR₉, COOR₉, CONHR₉ or CSNHR₉, whereby R₉ denotes (C₁-C₄)alkyl;
R₅ designates hydrogen, (C₁-C₄)alkyl, OH, (C₁-C₄)alkoxy, (C₁-C₂)alkoxy partly or fully fluorinated, trifluoromethyl, halogen or OX;
R₆ designates Me, halogen, (C₁-C₄)alkoxy, (C₁-C₂)alkoxy partly or fully fluorinated, SMe or SEt;
n is 1 or 2;
R₃′ and R₅′ each independently designate OH, Me, Et, OMe, OMe partly or fully fluorinated, trifluoromethyl or halogen;
U designates N or CR₂′, whereby R₂′ denotes hydrogen, (C₁-C₄)alkyl, (C₁-C₄)alkoxy, trifluoromethyl or halogen;
V designates N or CR₄′, whereby R₄′ denotes hydrogen, (C₁-C₆)alkoxy, (C₁-C₄)alkoxy partly or fully fluorinated, (C₁-C₆)alkyl, OH, trifluoromethyl, halogen or OX;
W designates N or CR₆′, whereby R₆′ denotes hydrogen, (C₁-C₄)alkyl, (C₁-C₄)alkoxy, trifluoromethyl or halogen;
wherein OX designates a group capable of conferring a prodrug property, said group being selected among phosphate derivatives, ester derivatives, carbonate derivatives and/or linked poly(ethylene glycols) derivatives; and pharmaceutically acceptable salts thereof;
with the provision that the compound of general formula (I) is not:
1-(3,5-dichloro-2-methoxyphenyl)-6-ethoxy-1,2,3,4-tetrahydroisoquinoline,
1-(3,5-dichloro-2-methoxyphenyl)-6-methoxy-1,2,3,4-tetrahydroisoquinoline,
1-(4-methoxy-3,5-dimethylphenyl)-5-ethoxy-6-methoxy-1,2,3,4-tetrahydroisoquinoline,
1-(4-methoxy-3,5-dimethylphenyl)-5,6-dimethoxy-1,2,3,4-tetrahydroisoquinoline,
1-(4-methoxy-3,5-dimethylphenyl)-6-ethoxy-1,2,3,4-tetrahydroisoquinoline,
1-(4-methoxy-3,5-dimethylphenyl)-6-methoxy-1,2,3,4-tetrahydroisoquinoline,
1-(3-chloro-2-methoxy-5-methylphenyl)-5-ethoxy-6-methoxy-1,2,3,4-tetrahydroisoquinoline,
1-(3-chloro-2-methoxy-5-methylphenyl)-5,6-dimethoxy-1,2,3,4-tetrahydroisoquinoline,
1-(3-chloro-2-methoxy-5-methylphenyl)-6-ethoxy-1,2,3,4-tetrahydroisoquinoline,
1-(3-chloro-2-methoxy-5-methylphenyl)-6-methoxy-1,2,3,4-tetrahydroisoquinoline,
and with the further provision that the compound of general formula (I) is not a compound according to the following general formula: 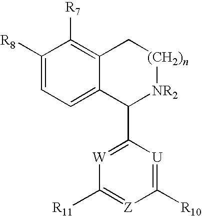
wherein
R₂ designates hydrogen, Me, Et, CHO, CN, OH, OMe, COR₉, COOR₉, CONHR₉ or CSNHR₉, whereby R₉ denotes (C₁-C₄)alkyl;
R₇ designates hydrogen, (C₁-C₄)alkyl, OH, (C₁-C₄)alkoxy, OCF₃, trifluoromethyl or halogen;
R₈ designates Me, (C₁-C₄)alkoxy, OCF₃, SMe or SEt;
n is 1 or 2;
R₁₀ and R₁₁ each independently designate OH, Me, Et, OMe, OCF₃, trifluoromethyl or halogen;
U designates N or CR₂′, whereby R₂′ denotes hydrogen, (C₁-C₄)alkyl, (C₁-C₄)alkoxy, trifluoromethyl or halogen;
Z designates N or CR₁₂, whereby R₁₂ denotes hydrogen, (C₁-C₆)alkoxy, (C₁-C₆)alkyl, OH, trifluoromethyl or halogen;
W designates N or CR₆′, whereby R₆′ denotes hydrogen, (C₁-C₄)alkyl, (C₁-C₄)alkoxy, trifluoromethyl or halogen;
and pharmaceutically acceptable salts thereof.

2. The compound according to claim 1, wherein R₄ is H, OH, NH₂, amino(C₁-C₃), amino(C₁-C₃)dialkyl, CH₂OH, COOCH₃, OCOOCH₃, methyl or Et.

3. The compound of claim 1, having the following general formula (II): 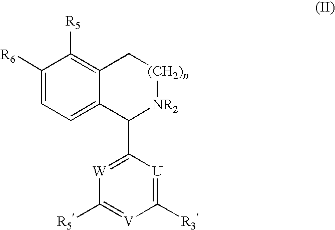
wherein at least one OX group is present in R₅ and/or R₄′ thereby conferring a prodrug property to the compound of formula (II); and pharmaceutically acceptable salts thereof.

4. The compound according to claim 1, wherein R₂ designates Me, OH, CN, CHO, COR₉ or COOR₉.

5. The compound according to claim 4, wherein R₂ designates Me, CN, CHO or COMe.

6. The compound according to claim 1, wherein R₅ designates hydrogen, Me, OMe, halogen, OH or OX.

7. The compound according to claim 1, wherein R₆ designates OCHF₂, OCH₂CF₃, OMe or OEt.

8. The compound according to claim 1, wherein R₅ designates OX, OH, hydrogen or OMe; and R₆ designates OCHF₂, OCH₂CF₃, OMe or OEt.

9. The compound according to claim 1, wherein R₃′ and R₅′ each independently designate chloro, bromo, Me, OMe or OCHF₂.

10. The compound according to claim 1, wherein R₃′ and R₅′ are identical; or R₃′ designates chloro or bromo, and R₅′ designates OMe.

11. The compound according to claim 9, wherein R₃′ and R₅′ designate both chloro, both bromo or both OCHF₂.

12. The compound according to claim 1, wherein U and W designate CH and V designates CR₄′.

13. The compound according to claim 12, wherein R₄′ designates hydrogen, OH, chloro, bromo, Me, OMe, OCHF₂ or OX.

14. The compound according to claim 1, wherein R₃′, R₄′ and R₅′ designate OMe; or R₃′ designates chloro and R₄′ and R₅′ designate OMe; or R₄′ designates hydrogen and R₃′ and R₅′ designate both chloro, both bromo or both OCHF₂.

15. The compound according to claim 1, which is the (R)- or (S)-enantiomer.

16. The compound of claim 1, wherein preferred carbonate derivatives are —OCOOCH₃, —OCOOC₂H₅, —OCOOPropyl, —OCOOIsopropyl, —OCOOBu, —OCOO(m-COONa-Ph), —OCOOCH₂CH₂COONa, —OCOOCH₂CH₂N(CH₃)₂.

17. The compound according to claim 1, wherein preferred phosphate derivatives are (1R)-1-(3,4,5-trimethoxyphenyl)-2-formyl-5-(dihydrogen phosphate)-6-methoxy-1,2,3,4-tetrahydroisoquinoline, (1R)-1-(3,5-dichlorophenyl)-2-formyl-5-(dihydrogen phosphate)-6-difluoromethoxy-1,2,3,4-tetrahydroisoquinoline and (1R)-1-[3,5-dichloro-4-(dihydrogen phosphate)phenyl]-2-formyl-6-difluoromethoxy-1,2,3,4-tetrahydroisoquinoline and their corresponding 6-(2,2,2-trifluoroethoxy), 2-cyano, and 2-acetyl derivatives, and pharmaceutically acceptable salts thereof.

18. The compound according to claim 1, wherein pharmaceutically acceptable salts are produced from acidic inorganic or organic compounds, or alkaline inorganic or organic compounds.

19. The compound as defined in claim 1, for use as a medicament.

20-21. (canceled)

22. A method of treatment or prophylaxis of a disease in which down-regulation or inhibition of the expression or function of the IGF-1 receptor is beneficial, in a subject in need thereof, comprising administering to said subject the compound of claim 1 in an amount which is effective in down-regulating or inhibiting the expression or function of the IGF-1 receptor.

23. The method of claim 22, wherein the disease is selected from cell proliferate diseases such as cancer, atherosclerosis, restenosis, inflammatory diseases such as psoriasis, autoimmune diseases such as rheumatoid arthritis, and transplant rejection.

24. A pharmaceutical composition comprising the compound of claim 1, and a pharmaceutically acceptable adjuvant, diluent or carrier.

25. Articles containing the compound of claim 1, and a chemotherapeutic agent, as a combination for the simultaneous, separate or successive administration in the therapy of a disease in which down-regulation or inhibition of the expression or function of the IGF-1 receptor is beneficial.

26. Use of the compound of claim 1, as a pharmacological tool in the development and standardization of in vitro and/or in vivo test systems for the evaluation of the effects of inhibitors of cell cycle activity in laboratory animals.
